(19) United States
(12) Patent Application Publication
(10) Pub. No.: US 2026/0345215 A1
(43) Pub. Date: Nov. 5, 2026

(54) CATHETER SEGMENT AND CATHETER COMPRISING SUCH SEGMENT

(71) Applicant: VascoMed GmbH, Binzen (DE)

(72) Inventors: Ralf Kaufmann, Loerrach (DE); Wolfgang Geistert, Rheinfelden (DE)

(73) Assignee: VascoMed GmbH, Binzen (DE)

(21) Appl. No.: 19/486,816

(22) PCT Filed: May 23, 2024
(86) PCT No.: PCT/EP2024/064266

§ 371 Date: Nov. 21, 2025

(30) Foreign Application Priority Data
Jun. 7, 2023 (EP) 23177882.0

(51) Int. Cl. A61B 18/00; A61M 25/00; A61M 25/01

(52) U.S. Cl. CPC A61B 18/00; A61M 25/0012; A61M 25/0045; A61M 25/005; A61M 25/0147; A61B 2018/00172; A61B 2018/00184; A61B 2018/00577

(57) ABSTRACT

The invention refers to a catheter segment of a catheter allowing steering in an asymmetrical, symmetrical, bi-directional and/or multidirectional manner, wherein the catheter segment extends along a longitudinal axis and comprises a tubular outer layer and a flexible reinforcing structure located within the tubular outer layer, wherein the catheter segment further comprises a first insert and at least one second insert, wherein each insert is attached to or integrally formed with the reinforcing structure, wherein each insert extends radially from the reinforcing structure through a neutral bending axis of the catheter segment and comprises at least one longitudinally extending guide structure for guiding a steering wire, wherein the first insert and the at least one second insert are located at predefined longitudinal positions of the catheter segment with a predefined longitudinal spacing. The invention further refers to a respective catheter and a manufacturing method of such catheter segment.

### CROSS-REFERENCE TO RELATED APPLICATIONS

This application is the United States National Phase under 35 U.S.C. § 371 of PCT International Patent Application No. PCT/EP 2024/064266, filed on May 23, 2024, which claims the benefit of European Patent Application No. 23177882.0, filed on Jun. 7, 2023, the disclosures of which are hereby incorporated by reference herein in their entireties.

### TECHNICAL FIELD

The invention relates to a bendable catheter, a catheter segment and a method of manufacturing such catheter segment.

### BACKGROUND

Catheters are medical devices with a broad range of functions. In many cases they comprise a handle at its proximal end and an elongated catheter shaft extending distally from this handle. Catheters may be inserted into a body cavity, duct, vessel, brain, skin or adipose tissue. They allow treatment and/or examination of a patient's tissue or bodily fluid, for example by means of electrodes located in a functional section of the catheter. Additionally or alternatively, catheters may be used to deliver and/or retrieve an implant to/from a target location of the patient's body. One example for a catheter comprising electrodes is an ablation catheter that may be used for performing cardiac ablation procedures, such as, but not limited to, pulmonary vein isolation (PVI), persistent atrial fibrillation ablation, or ventricular tachycardiac ablation. Alternatively or additionally, such catheter may allow examination of the patient's tissue using an electrical field.

Catheter such as the types mentioned above may comprise at least one steerable section located, for example, at its distal end. The steerable section allows steering of a functional catheter section, usually by means of at least one steering wire, such that it is positioned at a specific treatment or examination location within the patient's body. To precisely move the respective functional catheter section into different positions it is desirable to provide a steerable section that can be steered in an asymmetrical, symmetrical, bi-directional and/or multidirectional manner.

Document EP 0 566 724 B1 discloses three embodiments realizing an asymmetric bidirectional deflection of a distal catheter segment. The first embodiment is based on a leaf spring, to which a pull wire is attached on each side. Each pull wire is attached to its own turntable in the handle and said turntables are rotated coaxially about the same angle by a swivel lever. The deflection asymmetry is created by different shapes of the turntables and the resulting different tension and relief paths of the pull wires. An asymmetry of the deflection is created in the second embodiment by different distances of the attachment points of the pull wires from the distal end of the leaf spring. In the third embodiment, the asymmetry of the deflection is created by unilaterally stiffening elements on the leaf spring. In the first embodiment, the asymmetry of the deflections due to the shape of the turntables results only in a difference in the length of the deflection curve. The asymmetry of the deflections due to the position of the attachment points at the leaf spring leaves for one of the two deflections a straight distal (i.e. non-deflected) catheter segment and therefore does not make optimal use of the possibilities of “reach” shortening and “circle” formation. Asymmetry of deflection due to locally varying stiffnesses of the leaf spring on both sides results in asymmetric deflection shapes, but requires additional force for actuating the pull wires and reduces the sheath flexibility of the distal catheter segment.

From document WO 2022/087477 A1 catheter sheath extending lengthwise from a proximal end to a distal end along a sheath axis is known, wherein the catheter sheath comprises a central lumen extrusion having multiple layers including an inner layer defining a central lumen, a reinforcing structure surrounding the inner layer, and an outer layer surrounding the reinforcing structure in this order substantially concentric with the sheath axis. Further, a plurality of rings is arranged on the outer layer of the central lumen extrusion, wherein the plurality of rings is located at a predetermined distance from each other in a direction from the distal end towards the proximal end; and wherein the central lumen extrusion is bonded and/or pressure fit to one or more of the plurality of rings. In one embodiment explained in this document, the rings include guide rings comprising wire-guiding conduit arranged substantially parallel to and equidistant from the sheath axis; wherein at least one wire-guiding conduit in each guide ring includes at least one control wire slideably arranged along the length of the central lumen extrusion, a distal end of the at least one control wire being attached to an steerable segment of the steerable section and a proximal end of the at least one control wire configured to be mechanically connected to an actuator unit. For more complex bending of such catheter sheath a high number of driving wires is necessary thereby introducing complexity and increasing the outer diameter of the catheter sheath.

### SUMMARY

Accordingly, it is an object of the invention is to provide a catheter with a functional catheter section that can be steered in an asymmetrical, symmetrical, bi-directional and/or multidirectional manner, that is at the same time mechanically robust and has a small outer diameter. In addition, the aim is to have a catheter section that can withstand mechanical transverse and torsional loads in curved or deflectable sheaths to the same extent as known steerable catheters with multi-lumen tubing, which have the disadvantage of a very small effective lumen in the distal shaft region. Accordingly, it is a further object of the invention to provide a simple and cost-effective manufacturing method for such catheter.

The above object is solved by a catheter segment with the features of claim 1, a catheter having the features of claim 12 and a manufacturing method having the features of claim 14.

In particular, the object is solved by catheter segment of a catheter for treatment and/or examination of a patient's tissue or bodily fluid and/or for delivery and/or retrieval of an implant, wherein the catheter segment extends along a longitudinal axis and comprises a tubular outer layer and a flexible reinforcing structure located within the tubular outer layer, wherein the catheter segment further comprises a first insert and at least one second insert, wherein each insert is attached to or integrally formed with the reinforcing structure, wherein each insert extends radially within the reinforcing structure through a neutral bending axis of the catheter segment and comprises at least one longitudinally extending guide structure for guiding a steering wire, wherein the first insert and the at least one second insert are located at predefined longitudinal positions of the catheter segment with a predefined longitudinal spacing.

The inventive catheter segment comprises a flexible multilayer member comprising the tubular outer layer and the inner flexible reinforcing structure (also referred to as backbone structure) that may be formed essentially tubular, as well. The reinforcing structure is described in detail below. The steering properties of the catheter segment when used with at least one steering wire are based on the flexible multilayer member on the one hand as well as on the position and spacing of the single inserts located within the multilayer member and guiding the at least one steering wire on the other hand.

The tubular outer layer provides a flexible shell layer protecting the inner elements of the catheter segment. It may consist of or comprise at least one flexible plastic material of the group comprising thermoplastic Polyurethanes (TPU), Polyether-block-amides (PEBA), Silicone elastomers, and similar elastic materials.

The reinforcing structure (backbone structure, or short backbone) is a flexible, kink-resistant and essentially tubular structure (with cutouts). The reinforcing structure may comprise in one embodiment several web-like sections and several alternately arranged ring-like sections, for example, manufactured from a tube by laser cutting (i.e., cutting out wall sections). Depending on the desired flexibility, the dimensions of the ring-like sections and web-like sections are chosen as well as their sequence. For example, the web-like sections may comprise webs extending in longitudinal direction but vary with regard to their position along the circumference. The webs of one web-like section may connect two neighboring ring-like sections. For example, one ring-like section alternates with one web-like section having two or three parallel webs (the webs being parallel to the longitudinal axis of the catheter segment), wherein neighboring web-like section may comprise webs that are located at different positions along the circumference. In one embodiment, the web-like and ring structures are designed in such a way that the backbone bends almost equally in each direction when the same transverse force (radial force) is applied, i.e. there is largely isotropic bending flexibility. For example, the number and width of the webs, as well as the width of the ring-like sections or their material, are the key factors with regard to the bending flexibility of the catheter segment.

In one embodiment, the reinforcing structure is designed in such a way that bending by external forces cannot lead to contact, or collision of the edges of neighboring ring sections. Such collisions could cause leverage effects in the backbone structure that may plastically deform it or cause fracture damage. The length of the webs and the support provided by steering wires guided by the inserts is decisive with regard to this aspect.

Furthermore, in one embodiment, the width of the webs and its number is chosen such that the backbone structure can absorb the greatest possible torsional moments even in the curved state. This is supported by the steering wires counteracting torsion-induced shortening of the structure.

In one further embodiment, the widths of the webs and ring sections are chosen such that the backbone structure does not collapse under the tensile force of the steering wires.

Additionally, the above properties of the reinforcing structure depend on the material of this structure. For example, the reinforcing structure consists of or comprises at least one material of the group comprising an elastic plastic material, steel and nickel titanium alloy.

The above catheter segment allows steering of a functional catheter section in an asymmetrical, symmetrical, bi-directional and/or multidirectional manner depending on the position and detailed design of the insert as it is explained in the following. Further, the inserts can be modular-like arranged or provided within the reinforcing structure specifically for each desired steering type. The catheter segment, particularly the size, design and location of the insert may be created such that its structure is sufficiently robust against high mechanical transverse and torsional loads, such as those that can act on the distal region of the catheter during catheter treatment due to bent or deflectable long sheaths. Additionally, the inventive catheter segment provides useful small cross-sections and cavities for such elements like sensors, electrical leads, actuators and/or media lines that are necessary for the respective medical purpose of the catheter/catheter segment.

According to the invention, the insert is a component that extends within the reinforcing structure, in particular between opposite inner walls of the reinforcing structure and through the neutral bending axis of the catheter segment. Its form is comparable with a fixed spoke (in one direction) and additionally extends in longitudinal direction of the catheter segment (second direction).

In one embodiment, the guide structure for the at least one steering wire is a longitudinally extending through hole or groove within/along the insert. Such guide structure is easy to realize. In one embodiment, each insert comprises two parallel or inclined guide structures for guiding two steering wires or three parallel or inclined guide structures for guiding three steering wires. In one embodiment, the guide structure(s) is/are parallel to the longitudinal axis of the catheter segment.

In one embodiment, one guide structure of the first insert and/or of the at least one second insert runs along the neutral bending axis of the catheter segment. Such guide structure does not provide bending of the catheter segment if the steering wire is pulled. Such guide structure is therefore used for a steering wire that provides bending at another section of the catheter or that is needed for other functional elements such as scissors or grabbing/releasing element. Such functional element may be provided at the distal end of the respective catheter segment or catheter (for which the catheter segment is used). Additionally or alternatively, a steering wire (also referred to as pull wire) for triggering an action (e.g. a sensor release) is guided over the entire longitudinal dimension of the catheter or catheter segment along the neutral bending axis. Alternatively, one single steering wire may proximally be guided within a guide structure that runs along the neutral bending axis. Towards the distal end of the catheter or catheter segment, the steering wire is guided step-by-step via appropriately arranged guide structures in the inserts at an increasing distance from the neutral bending axis. Accordingly, different inserts arranged along the longitudinal axis of the catheter segment may comprise one guiding structure or more than one guiding structures at the same and/or different positions.

In one embodiment, the catheter segment further comprises at least one incompressible tubular element located between and directly adjacent to the first insert and the neighbouring second insert guiding at least one steering wire between these first and second inserts. The tubular element may be flexible and may consist of or comprise at least one material of the group comprising Polyimides (PI), braid reinforced Polyimides, Polyamides (PA), steel compression coils, or similar tube structures. The tubular element prevents the backbone structure from compressing due to the forces in the tension wires that are generated to trigger its deflections or action, the neighbouring inserts are kept at a distance by the incompressible supporting tubular elements. The counterforce (compressive force) in the respective tubular element is transferred via the inserts to the backbone structure.

In one embodiment, the first insert and the neighboring second insert are accommodated such that the radial extension (dimension) of the first insert is parallel to or arranged at a predefined angle to the radial extension of the second insert. In one embodiment the first insert and the second insert have a common longitudinal axis, wherein the radial extension is perpendicular or inclined with regard to the common longitudinal axis. In this embodiment, the angle is defined by a rotation about the longitudinal axis. If the radial extension of the first and second inserts is parallel, the catheter segment between both inserts bents within one common plane. In contrast, if the first insert and the neighboring second insert are arranged at a predefined angle pulling at the steering wire causes twisting of the catheter segment, particularly if one or several further inserts are arranged at an additional, predefined distance from the first and second inserts.

In one embodiment, a longitudinal dimension (length L) of each insert is equal to or greater than 0.75 times its radial dimension (height H). Additionally, the third dimension (width B) may be much smaller compared with the length L and the height H. Such dimension of the insert has proven to be of sufficient size to absorb torsional and transverse loads when deflected, securely guiding at least one steering wire and leaving some open volume for sensors, electrical leads, actuators and/or media lines.

In one embodiment, each insert is attached to (and supported by) the inner reinforcing structure by a form-locking connection and/or by a force-locking connection and/or by a substance-to-substance bond. Thereby, the insert may be attached at a desired location within the multilayer structure (i.e. within the reinforcing structure) and under a desired angle with regard to the neighboring component, i.e. modularly attached, to provide the desired deflection properties. In one embodiment, the inner reinforcing structure comprises a guide structure for the steering wire, for example a projecting eyelet (lug) that projects into the open inner lumen of the reinforcing structure, cooperating with one guide structure or one recess of the corresponding insert such that it forms a form-locking connection, e.g. a guide structure formed as a groove. I.e. the outer form of the eyelet corresponds to the inner form of the groove or recess so that the eyelet can be moved into and accommodated within the guide structure. After providing this arrangement, the position of the eyelet and the groove is fixed by the steering wire running through the eyelet and along the groove. The insert can be rotated within the inner lumen of the reinforcing structure so that the eyelet is introduced into the recess or, if applicable by a further translational movement into the groove of the insert forming a force-locking connection. Additionally, thereby the guiding structure (groove) and the through hole (opening) of the eyelet are aligned such that the steering wire runs along the groove and through the eyelet.

The insert may consist of or comprise at least one material of the group comprising Polyether ether ketone (PEEK), Polyoxymethylene (POM), other hard plastic material, metal material, ceramic material and glass material. The insert may be a laser-cut, mechanically machined sintered or micro injection casted element.

The above object is also solved by a catheter having a handle at its proximal end, an elongated catheter shaft attached to and located distally from the handle, at least one steering wire that is attached to and controlled by the handle, wherein a longitudinal section of the catheter shaft is formed by the catheter segment as described above, wherein the at least one steering wire extends within an inner lumen of the reinforcing structure and is guided by the at least one guiding structure of said inserts of the catheter segment.

The handle of the catheter is fixed to the catheter shaft and used to introduce the catheter shaft into the patient's body. Further, the handle comprises an actuator to pull and/or push the at least one steering wire connected to the actuator to steer the catheter shaft and, if applicable, its functional section. The steering wire may extend at least partly outside the catheter shaft but along the at least one catheter segment within its respective reinforcing structure.

As indicated above, the catheter segment forms a section of the catheter shaft, wherein one such catheter segment or two or more than two catheter segments accommodated one after the other along the longitudinal axis of the catheter shaft may be used. The catheter shaft may further comprise at least one functional section adjacent to one catheter segment comprising the inserts. Such functional section may comprise, for example, electrodes (e.g. ring electrodes) for therapeutic use (e.g. ablation) or electrophysiological diagnostics.

The steering wire may consist of or comprise at least one material of the group comprising steel, Nickel Titanium alloy, Ultra-high-molecular-weight polyethylene (UHMWPE), or similar smooth high strengths materials. The steering wire is attached to the catheter shaft at its distal end, for example by gluing or welding, and/or attached to an actuator at its proximal end.

The catheter may be an ablation catheter having at least one electrode (head electrode and/or ring electrode) at the distal end of the catheter shaft. Alternatively, the catheter may be an implant or sensor deployment catheter, or a tissue, debris, or thrombus removal catheter or a catheter equipped with optical, mechanical or chemical diagnostic or therapeutic functions. The above object is further solved by a manufacturing method for a catheter segment as described above, wherein the insert is introduced into the reinforcing structure along its longitudinal direction, then rotated about its longitudinal axis until a form-locking connection is formed between the reinforcing structure and the insert and after that the steering wire is introduced into the guide structure. As indicated above, the form-locking connection may be provided, for example, by a projecting eyelet of the reinforcing structure and a groove forming a guide structure. The form-locking connection/the insert may additionally be secured against rotation by a steering wire running along a guide structure aligned with an opening of the eyelet.

### BRIEF DESCRIPTION OF THE DRAWINGS

The present invention will now be described in further detail with reference to the accompanying schematic drawing, wherein

FIG. 1 shows a first embodiment of an inventive catheter in a first state with no deflection of the distal end section in a perspective side view,

FIG. 2 depicts the embodiment of FIG. 2 in a second state with respect to a large deflection curve along the distal end section in a perspective side view,

FIG. 3 shows the embodiment of FIG. 2 in a third state with respect to a small deflection curve along the distal end section in a perspective side view,

FIG. 4 depicts the distal end sections of the embodiment of FIG. 1 in the states as shown in FIGS. 2 and 3 in comparison in side views,

FIG. 5 depicts the distal end section of the embodiment of FIG. 1 shown in the state of FIG. 2 in a partially sectioned side view,

FIG. 6 depicts the distal end section of the embodiment of FIG. 1 shown in the state of FIG. 3 in a partially sectioned side view,

FIG. 7 depicts a transverse section of the catheter segment of the embodiment of FIG. 1 in a bent state,

FIG. 8 shows the transverse section of the catheter segment of FIG. 7 in a straight state,

FIG. 9 depicts one insert of the catheter segment of the embodiment of FIG. 1 in a perspective side view,

FIG. 10 shows the insert of FIG. 9 within one portion of the catheter segment of the embodiment of FIG. 1 in a sectioned perspective side view,

FIG. 11 shows a second embodiment of one insert that can be used with another embodiment of a catheter in a perspective side view,

FIG. 12 depicts a third embodiment of one insert that can be used with another embodiment of a catheter in a cross section,

FIG. 13 shows a distal end section of a second embodiment of a catheter in a first (straight) state in a partially sectioned side view,

FIG. 14 depicts the view and embodiment of FIG. 13 in a second (deflected) state,

FIG. 15 shows the view and embodiment of FIG. 13 in a third (deflected) state, and

FIG. 16-21 depict the manufacturing steps of the catheter segment of FIG. 1 in perspective side views.

### DETAILED DESCRIPTION

The invention is explained in the following with regard to an ablation catheter 1 for intracardiac therapeutic use. It may similarly be used for any other catheter type as explained above.

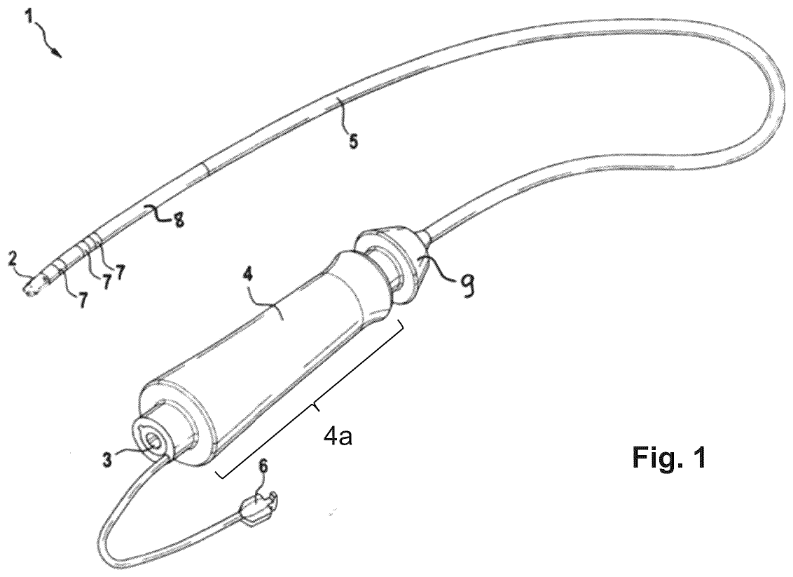

The ablation catheter shown in FIG. 1 to 3 comprises a handle 4 and a catheter shaft 5 attached to the distal end of the handle 4. The catheter shaft 5 comprises a distal section 8 with a head electrode 2 for application of the ablation energy. Three parallel ring electrodes 7 located proximal from the head electrode 2 at the distal section 8 are used for electrophysiological diagnostics. In this embodiment, the most distal end of the distal section 8 comprising the electrodes 2, 7 forms the functional section of the catheter shaft 5 of the catheter 1.

The handle 4 comprises a connection socket 3 for the electrical connection of the electrodes to a controller and a generator. The handle 4 further comprises a connection port 6 for flushing the catheter system, wherein the connection socket 3 and the connection port 6 are located at the proximal end of the handle 4.

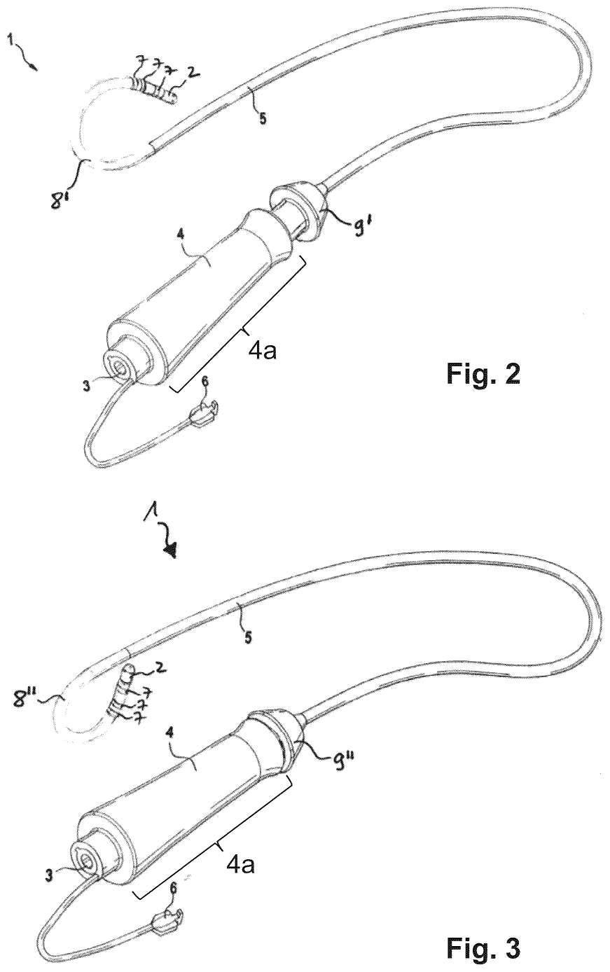

FIG. 1 shows the distal section 8 of the catheter shaft 5 in a straight state. The state of the distal section of the catheter shaft 5 shown in FIG. 2 is deflected (see reference number 8′) but with a greater radius than in the state of the distal section shown in FIG. 3 (see reference number 8″). To change the bending radius, a control button 9 provided at the distal end of the handle 4 is actuated. When the control button is pushed out (see reference number 9′ in FIG. 2) the bending radius is greater than when the control button is pushed in (see reference number 9″ in FIG. 3). To deflect the distal section 8 button 9 of the handle 4 is moved away from or towards to the central section 4a of the handle 4.

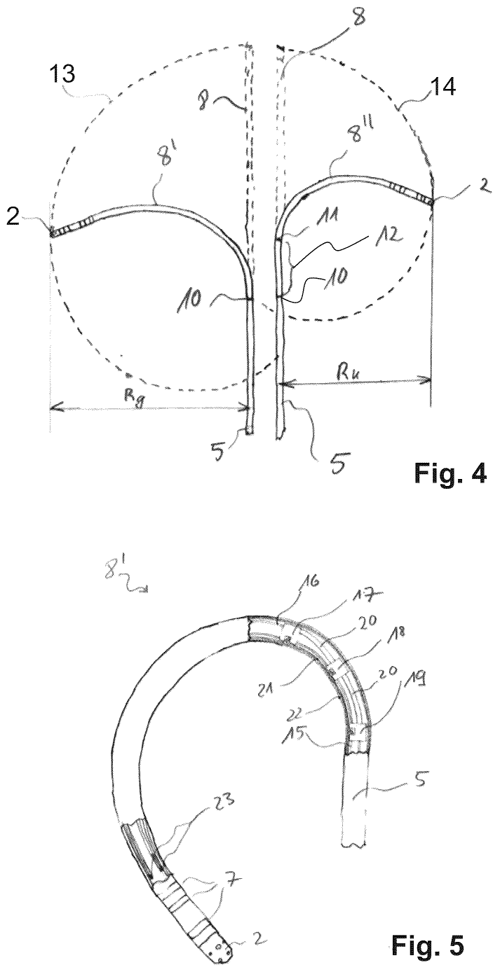

The different bending radii Rg and Rk are visualized in comparison in FIG. 4. On the left hand side, the greater bending radius Rg of FIG. 2 is shown, whereas the smaller bending radius Rk realized in FIG. 3 is depicted on the right hand side. The different bending radii/states are caused by bending or keeping straight a catheter segment 12 which forms the proximal end of the distal section 8. When a greater bending radius Rg is formed (reference number 8′), the catheter segment 12 is bent prior/during deflecting the distal section 8, whereas a smaller bending radius Rk is produced (reference number 8″) when the catheter segment 12 is kept straight during deflection of the distal section 8.

The catheter segment 12 extends in longitudinal direction between its proximal end 10 and its distal end 11 (see FIG. 4). The proximal end 10 forms the transition to the proximal section of the catheter shaft 5 that is not steerable and the distal end 11 forms the transition to the portion of the distal section 8 that can be deflected in a manner depending on the state of the catheter segment 12. In FIG. 4, the initial position of the distal section 8 (straight position) is shown with a dashed line. The dashed line 13 shows the possible deflection path of the head electrode 2 if the greater bending radius Rg is realized whereas the dashed line 14 visualizes the possible deflection path of the head electrode 2 if the smaller bending radius Rk is used.

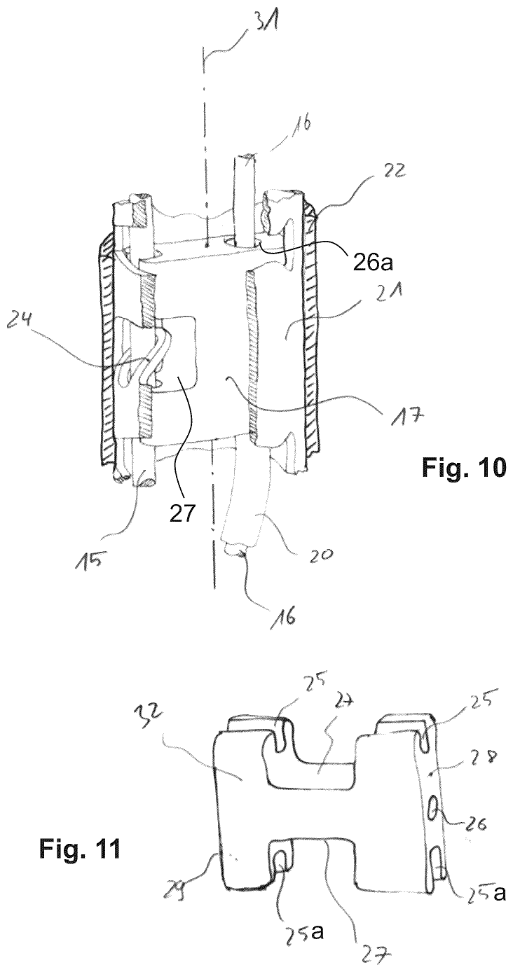

As indicated above, the different deflection behavior is caused by the catheter segment 12 which is shown in FIGS. 5 to 8 in more detail. The catheter segment 12 comprises a tubular outer layer 22, a flexible reinforcing structure (backbone structure) 21, a first insert 17, a second insert 18 and a third insert 19 located with a pre-defined longitudinal spacing along the longitudinal axis/neutral bending axis 31 of the tubular outer layer 22 within the inner lumen 33 of the reinforcing structure 21. Each of the longitudinal distance between the first insert 17 and the second insert 18 as well as between the second insert 18 and the third insert 19 is maintained by a respective incompressible tubular element 20 located between the respective neighboring inserts 17, 18 and 18, 19, wherein a second steering wire 16 is guided through the inserts 17, 18, 19 and the tubular elements 20. End faces 28, 29 running perpendicular to the longitudinal direction of inserts 17, 18, 19 form abutting surfaces for the tubular elements 20 (see FIG. 10).

One portion of the reinforcing structure 21 is depicted in FIG. 16. The reinforcing structure 21 may consist of a Nickel Titanium alloy and may comprise alternating web-like sections 21a and ring-like sections 21b. The web-like sections 21a may be produced, for example, by cutting out respective wand portions of the initial tube using a laser. As one can derive from FIG. 16, in the shown embodiment each web-like section 21a comprises two parallel webs 21c, wherein the parallel webs 21c of neighboring web-like sections 21 are shifted along the circumference of the reinforcing structure 21. Further, the ring-like section 21b comprises an eyelet/lug 24 projecting from the reinforcing structure 21 into the inner lumen 33 of this structure for accommodation of a first steering wire 15 and attachment of the first, second or third insert 17, 18, 19, respectively, as described in detail below.

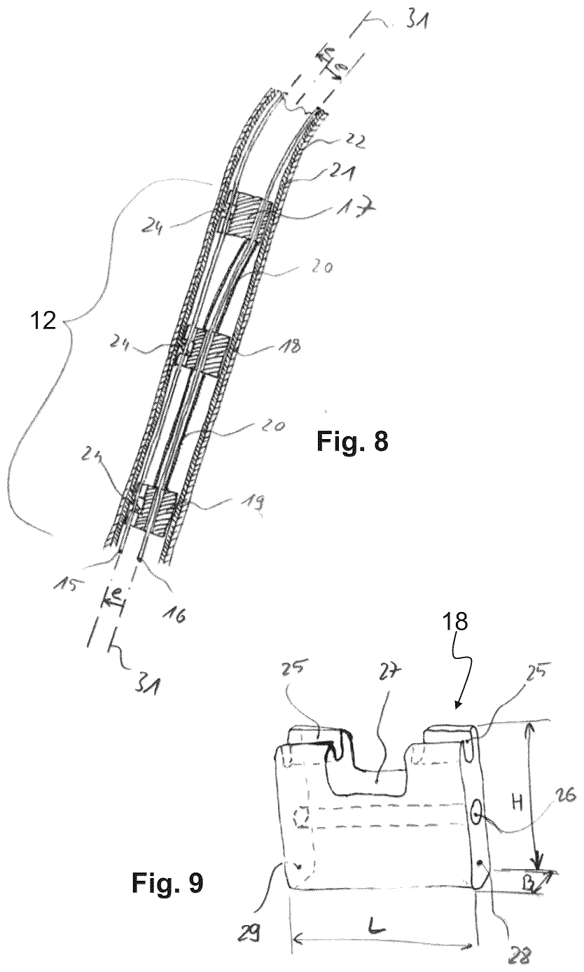

The flexible tubular outer layer 22 covers the reinforcing structure 21 and seals/protects the inner components of the catheter shaft. The outer layer consists, for example, of thermoplastic Polyurethanes (TPU), Polyether-block-amides (PEBA), Silicone elastomers, and similar elastic materials. The incompressible tubular element 20 is incompressible and maintains the distance of two neighboring inserts 17, 18, 19 not only in the straight state (see FIG. 8) but also in the curved/bent state of the catheter segment 12 (see FIG. 7). The tubular element 20 may consist, for example, of a Polyimide (PI), braid reinforced Polyimide, Polyamide (PA), steel compression coil, or a similar tube structure.

Within inner lumen 33 of the multilayer structure comprising the tubular outer layer 22 and the reinforcing structure 21 the catheter segment 12 comprises the first, second and third inserts 17, 18, 19. The structure of the first insert 17 is shown in greater detail in FIG. 10 and of the second and third inserts 18, 19 in FIG. 9.

Each insert 17, 18, 19 has basically a cuboid shape. When located within the reinforcing structure 21, its longitudinal dimension (L, see FIG. 9) is at least 0.75 times its radial dimension (H). The third dimension (thickness B) is significantly smaller than the longitudinal dimension L and the radial dimension H. For example, with regard to the shown embodiment, the longitudinal dimension is L=2 mm . . . the radial dimension H=1,8 mm and the third dimension B=0,5 mm.

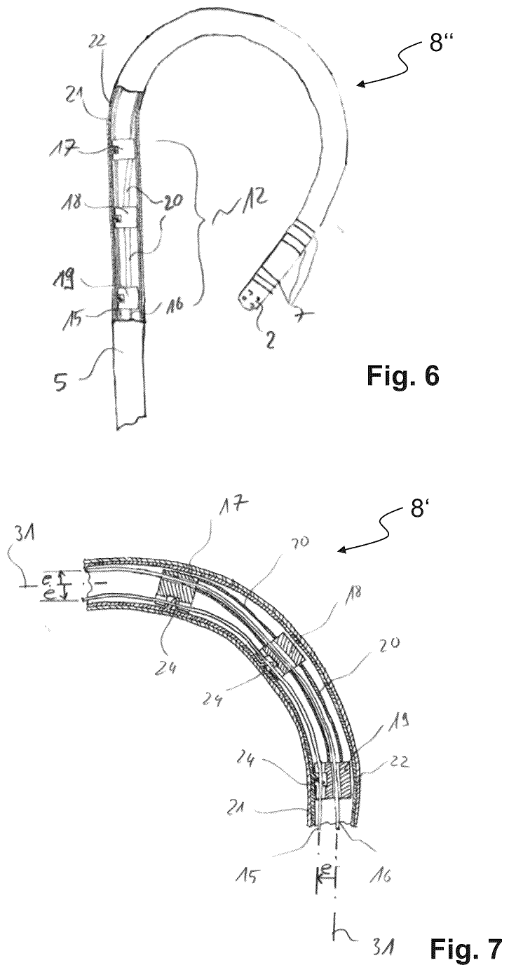

The third and second inserts 18, 19 comprise a guiding structure in the form of a groove 25 running along one longitudinally extending side of the respective insert 18, 19. Additionally, they comprise a guiding structure in the form of a through hole 26 extending in longitudinal direction when located in the catheter segment 12. In addition, the through hole 26 runs along the neutral bending axis/longitudinal axis 31 when the insert 18, 19 is accommodated in the catheter segment 12 (see FIGS. 7 to 10). The groove 25 has a (radial) distance e (see FIG. 8) from the longitudinal axis 31. In contrast, the first insert 17 comprises two guiding structures 25, 26a that have a (radial) distance from the neutral bending axis/longitudinal axis 31 as it is depicted in FIG. 9, for example. The groove 25 of the first, second and third inserts 17, 18, 19 guide the first steering wire 15 when located within the catheter segment and the through hole 26 guides the second steering wire 16. The first steering wire 15 and the second steering wire 16 are attached (e.g. by laser welding) to the reinforcing structure 21 at their distal ends as shown in FIG. 5 proximally from the functional section of the catheter shaft 5. Because the second steering wire 16 runs along the neutral bending axis 31 over most of the catheter segment 12, pulling the second steering wire 16 does not bent the catheter segment 12 during deflection (see FIGS. 6 and 8), whereas pulling the first steering wire 15 does (see FIGS. 5 and 7). Accordingly, if the control button 9 is pushed in (reference number 9″ of FIG. 3) pulling the first steering wire 15 is prevented and deflection of the distal section 8 of the catheter shaft is provided by the second steering wire 16, only. If the control button 9 is pushed out (see reference number 9′ of FIG. 2) deflection is provided by pulling the first steering wire 15 to bend the catheter segment 12 thereby realizing a greater bending radius Rg. E. g. the button contains a toothed rack which turns a balance-lever. The steering wires are connected with the opposite ends of the balance lever. This is also caused by the fact that between the first insert 17 and the second insert 18 the second steering wire 16 is led radially outward so that pulling the second steering wire 16 causes a deflection of the catheter shaft 5 only distal from the catheter segment 12.

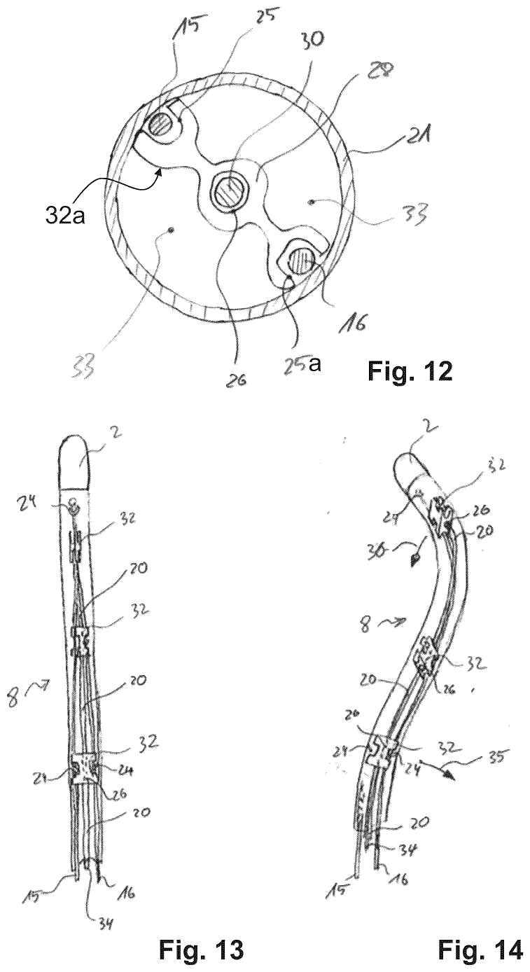

FIGS. 11 and 12 show further embodiments of inserts 32 and 32a having three guiding structures, namely two grooves 25, 25a running along two opposite side surfaces of the inserts 32, 32a and one central through hole 26 running along the neutral bending axis 31. The insert 32a shown in FIG. 12 comprises a smaller third dimension (B) between the guiding structures 25, 26 to provide a greater lumen 33 between the reinforcing structure 21 and the insert 32a for accommodation of functional components such as electrical leads. A third steering wire 30 runs along the central through hole 26 as shown in FIG. 12.

In one embodiment, the third steering wire 30 may run along the whole distal section 8 along the neutral bending axis. Such guide 30 wire may actuate a scissors or similar functional component accommodated at the proximal end. Pulling or pushing such steering wire 30 does not cause any deflection of the distal section 8 of the catheter shaft 5.

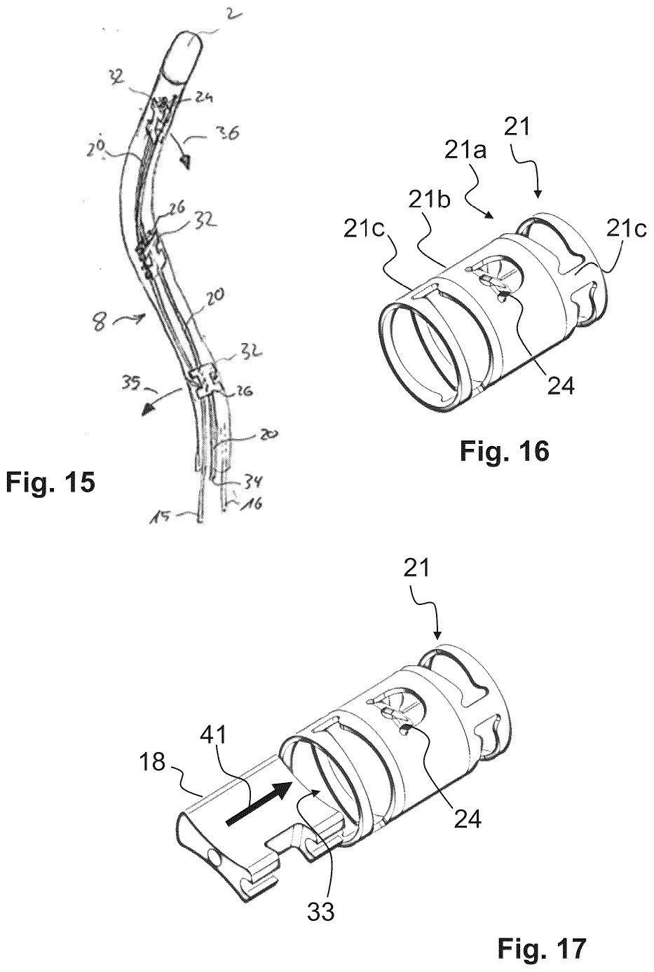

FIGS. 13 to 15 show an embodiment that illustrates how a three-dimensional bending curve of the distal section 8 is realized. In this embodiment, the catheter segment 12a comprises three inserts 32 as shown in FIG. 11 and FIG. 12 in detail. These inserts are located such within the reinforcing structure 21 that the radial extension (H) of neighboring inserts 32 are arranged at a predefined angle about the common longitudinal axis/neutral bending axis. Additionally, all inserts 32 have a central wire 34 extending along the longitudinal axis and through the through hole 26 that stabilizes the inserts 32 when the first and second steering wires 15, 16 are deflected. Due to the twisted accommodation of the inserts 32 a complex three-dimensional deflection of the distal section 8 of the catheter shaft can be provided when pulling the steering wires 15, 16 as shown in FIGS. 14 and 15.

In FIG. 14 the second steering wire 16 is pulled, the first steering wire 15 is omitted for clarity reasons. The deflection directions during pulling the second steering wire 16 are visualized by arrows 35 and 36. The tubular elements 20 accommodating the central steering wire 34 prevent collapse of the reinforcing structure 21 even under high tensile forces or external loads.

FIG. 15 shows the deflection if the first steering wire 15 is pulled. The second steering wire 16 is omitted for sake of clarity. Such traction results in opposite deflections (see arrows 35 and 36) compared to FIG. 14.

The skilled person easily understands that based on use and arrangement (distances and angular arrangement) of inserts (such as inserts 17, 18, 19, 32), for example, consisting of PEEK, as well as on different arrangement of guiding structures within used inserts, a great variety of deflection shapes of a predefined section (e.g. of distal section 8 or of any other predefined further proximal section) can be created and combined in one catheter.

In addition, the arrangement can be easily manufactured. FIGS. 16 to 21 show the assembly of an insert (e.g. second insert 18) within the reinforcing structure (backbone) 21 wherein only one portion of the reinforcing structure 21 is shown as indicated above.

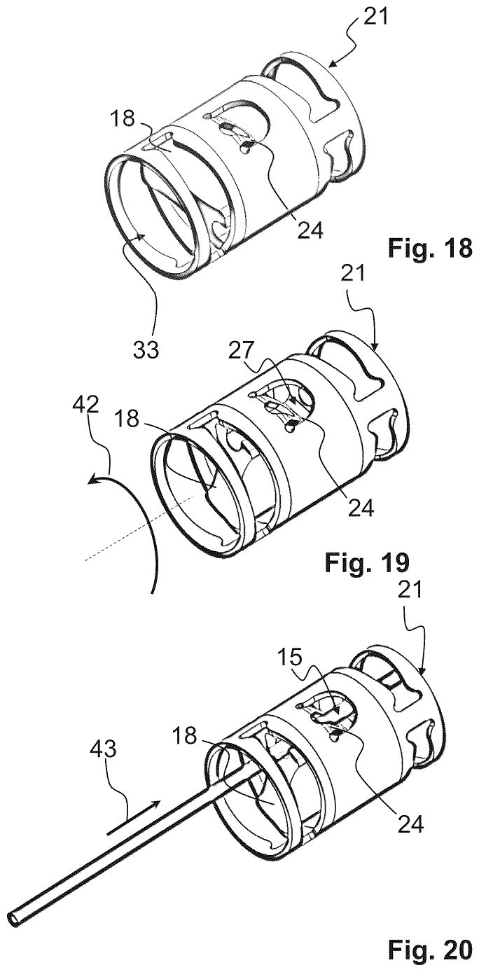

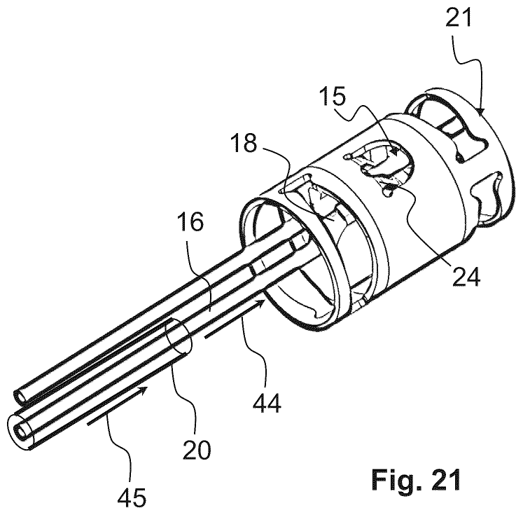

In the first step, the reinforcing structure 21 is provided as shown in FIG. 16. Then, without colliding with the projecting eyelet 24, the second insert 18 is moved into the inner lumen 33 of the reinforcing structure 21 (see arrow 41 of FIG. 17) until the recess 27 is positioned at the level of the tab 24 (see position shown in FIG. 18). The insert 18 is then rotated (see arrow 42 in FIG. 19) within the reinforcing structure 21 around its longitudinal axis until the eyelet 24 is located within the recess 27. As a result, a back and forth movement of the insert 18 is no longer possible. In the next step, the first steering wire 15 is introduced into the groove 25 of the insert 18 and through the eyelet 24 of the reinforcing structure 21 (see arrow 43 in FIG. 20). Hence, it is no longer possible to rotate the insert 18 within the reinforcing structure 21. It is completely fixed in the reinforcing structure 21. After that, the second steering wire 16 is introduced into the through hole 26 of the insert 18 (see arrow 44 of FIG. 21). In the final step, the tubular element 20 is guided over the second steering wire 16 all the way to the insert surface 28 forming a stop surface (see arrow 45 of FIG. 21).

Further inserts (e.g. inserts 17, 19, 32) may be successively threaded over the second steering wire 16 up to the support tube 20, rotated in the area of an eyelet 24 and secured against further turning by introducing the first steering wire 15.

As a final step, the outer tubular layer 22 is accommodated over the reinforcing structure 21 by overdrawing. Then, the steering wires as well as the catheter shaft 5 are mechanically connected with the handle 4 and its parts.

The invention creates a mechanically robust and at the same time flexible, steerable catheter segment with a reinforcing structure 21 cut from an elastic material (called “backbone”), which is integrated, for example, in therapy, diagnostic or implant delivery and retrieval catheters. The invention uses components in the form of inserts 17, 18, 19, 32, 32a and defines their interaction with the reinforcing structure 21. This interaction enables three-dimensional symmetrical and asymmetrical deflections of a section (e.g. a distal section 8, 8′, 8″) of the catheter shaft 5.

1. A catheter segment of a catheter, wherein the catheter segment extends along a longitudinal axis and comprises a tubular outer layer and a flexible reinforcing structure located within the tubular outer layer, wherein the catheter segment further comprises a first insert and at least one second insert, wherein each insert is attached to or integrally formed with the reinforcing structure, wherein each insert extends radially from the reinforcing structure through a neutral bending axis of the catheter segment and comprises at least one longitudinally extending guide structure for guiding a steering wire, wherein the first insert and the at least one second insert are located at predefined longitudinal positions of the catheter segment with a predefined longitudinal spacing.

2. The catheter segment of claim 1, wherein the guide structure is a longitudinally extending through hole or groove.

3. The catheter segment of claim 1, wherein each insert comprises two parallel or inclined guide structures for guiding two steering wires or three parallel or inclined guide structures for guiding three steering wires.

4. The catheter segment of claim 1, wherein one guide structure of the first insert and/or of the at least one second insert runs along the neutral bending axis of the catheter segment.

5. The catheter segment of claim 1, wherein the reinforcing structure comprises several web-like sections and several alternately arranged ring-like sections.

6. The catheter segment of claim 1, further comprising at least one incompressible tubular element located between and directly adjacent to the first insert and the neighboring second insert guiding at least one steering wire between these first and second inserts.

7. The catheter segment of claim 1, wherein the first insert and the neighboring second insert are accommodated such that the radial extension of the first insert is parallel to or arranged at a predefined angle to the radial extension of the second insert.

8. The catheter segment of claim 1, wherein a longitudinal dimension of each insert equal to or greater than 0.75 times its radial dimension.

9. The catheter segment of claim 1, wherein each insert is attached to the inner reinforcing structure by a form-locking connection and/or by a force-locking connection and/or by a substance-to-substance bond.

10. The catheter segment of claim 1, wherein the inner reinforcing structure comprises a guide structure for the steering wire, for example a projecting eyelet, cooperating with one guide structure or one recess of the corresponding insert.

11. The catheter segment of claim 10, wherein the through hole of the eyelet is aligned with a guiding structure.

12. A catheter for treatment and/or examination of a patient's tissue or bodily fluid, for example using an electrical field, having a handle at its proximal end, an elongated catheter shaft located distally from the handle, at least one steering wire that is attached to and controlled by the handle, wherein a longitudinal portion of the catheter shaft is formed by the catheter segment according to claim 1, wherein the at least one steering wire extends within an inner lumen of the reinforcing structure and is guided by the at least one guiding structure of said inserts of the catheter segment.

13. The catheter of claim 12, wherein the catheter is an ablation catheter.

14. A manufacturing method for a catheter segment of claim 1, wherein the insert is introduced into the reinforcing structure along its longitudinal direction, then rotated about its longitudinal axis until a form-locking connection is formed between the reinforcing structure and the insert and after that the steering wire is introduced into the guiding structure.
